(19) United States
(12) Patent Application Publication
(10) Pub. No.: US 2025/0057789 A1
(43) Pub. Date: Feb. 20, 2025

(54) STABLE ORAL LIQUID FORMULATIONS CONTAINING METOPROLOL OR SALTS THEREOF

(71) Applicant: RUBICON RESEARCH PRIVATE LIMITED, Thane West (IN)

(72) Inventors: Pratibha PILGAONKAR, Thane West (IN); Vaibhav DESHMUKH, Thane West (IN); Ashwini GADKARI, Thane West (IN)

(21) Appl. No.: 18/810,106

(22) Filed: Aug. 20, 2024

(30) Foreign Application Priority Data
Aug. 20, 2023 (IN) 202321046213

(51) Int. Cl. A61K 31/138; A61K 9/00; A61K 9/08; A61K 47/10; A61K 47/12; A61K 47/18; A61K 47/36

(52) U.S. Cl. CPC A61K 31/138; A61K 9/0053; A61K 9/08; A61K 47/10; A61K 47/12; A61K 47/18; A61K 47/36

(57) ABSTRACT

The present disclosure relates to stable oral liquid pharmaceutical formulations comprising metoprolol or pharmaceutically acceptable salts thereof, methods of manufacturing the same, as well as applications thereof including the use of the inventive formulations for treatment of angina, hypertension, and myocardial infarction.

### FIELD

The present disclosure relates to stable oral liquid pharmaceutical formulations comprising metoprolol or pharmaceutically acceptable salts thereof, one or more preservatives, one or more cosolvents, and water. The present disclosure further relates to stable oral liquid pharmaceutical formulations comprising metoprolol or pharmaceutically acceptable salts thereof, one or more preservatives, one or more chelating agents, one or more sweeteners, and water. The formulations of the present disclosure are palatable, alcohol-free, and sugar-free. The present disclosure further relates to method of treatment of angina, hypertension, and myocardial infarction comprising administering to the subject in need thereof stable oral liquid pharmaceutical formulation comprising metoprolol or pharmaceutically acceptable salts thereof, one or more preservatives, one or more chelating agents, one or more sweeteners, and water.

### BACKGROUND

Hypertension is a disease in which blood flows through blood vessels (arteries) at a higher-than-normal pressure. It is also known as high blood pressure. Approximately 1 in 3 American adults have high blood pressure. But only half of those people have their condition under control. Many people develop high blood pressure when they are in their late 30's or early 40's, and it occurs more frequently as people age. However, because of the obesity epidemic, more and more children are also developing high blood pressure. Early detection of high blood pressure is very important. Often referred to as the “silent killer” because it may show no symptoms, high blood pressure increases the risk for heart disease, heart failure, and stroke, among other things. As per the National Health and Nutrition Examination Survey 2017-March 2020 the percent of adults aged 18 and over with hypertension (measured high blood pressure and/or taking antihypertensive medication) was 48.1% in United States. Left unchecked, hypertension is considered a substantial risk factor for cardiovascular and other diseases including coronary heart disease, myocardial infarction, congestive heart failure, stroke and kidney failure.

Hypertension is classified as primary (essential) hypertension or secondary hypertension. Essential hypertension (also called primary hypertension, or idiopathic hypertension) is the form of hypertension that has no identifiable secondary cause. Primary hypertension tends to be familial and is likely to be the consequence of an interaction between environmental and genetic factors. Prevalence of essential hypertension increases with age, and individuals with relatively high blood pressure at younger ages are at increased risk for the subsequent development of hypertension. It is the most common type of hypertension. Secondary hypertension can be caused by drug or surgical interventions or by other systemic abnormalities.

Treatment of high blood pressure often starts with lifestyle changes, including decreasing salt in your diet, losing weight if necessary, stopping smoking, cutting down on alcohol use, and regular exercise. In addition to lifestyle changes, antihypertensive medications are often used to lower blood pressure. Therapeutic classes of antihypertensive drugs commonly used include alpha-adrenergic blockers, beta-adrenergic blockers, calcium-channel blockers, mineralocorticoid antagonists, diuretics, and angiotensin II receptor antagonists (ARB) or angiotensin-converting enzyme (ACE) inhibitors etc.

Metoprolol is a short-acting beta blocker that works specifically on the heart. It works by slowing down the heart rate and makes the heart more efficient at pumping blood around the body. Clinical pharmacology studies have demonstrated the beta-blocking activity of metoprolol, as shown by (1) reduction in heart rate and cardiac output at rest and upon exercise, (2) reduction of systolic blood pressure upon exercise, (3) inhibition of isoproterenol-induced tachycardia, and (4) reduction of reflex orthostatic tachycardia. Metoprolol is commonly employed as the succinate and tartrate salts.

Metoprolol tartrate is available in the form of tablets. LOPRESSOR® (metoprolol tartrate) tablets were approved prior to Jan. 1, 1982. Some geriatric or seriously ill patients may however find it difficult to swallow tablets. Furthermore, the prescribing information of LOPRESSOR® indicates that blood levels are likely to increase substantially in patients with hepatic impairment. Therefore, LOPRESSOR® should be initiated at low doses with cautious gradual dose titration according to clinical response. Additionally, for geriatric patients (>65 years), the LOPRESSOR® prescribing information indicates that a low initial starting dose may be used in elderly patients given their greater frequency of decreased hepatic, renal, or cardiac function, and of concomitant disease or other drug therapy.

For addressing such requirements in the patient population, a liquid dosage form that is palatable, stable, easy to swallow, convenient and which allows for dose titration to meet the needs of the patients is desirable.

U.S. Patent Publication No. 2009/0264535 discloses a liquid drug formulation for beta blockers, which is suitable for oral application in animals. Further, the United States Pharmacopoeia (USP) has a monograph for Metoprolol Tartrate Compounded Oral Solution. Such a compounded oral solution must be used within 60 days of the date on which it was compounded when stored at controlled room temperature or in a refrigerator.

What is needed are stable oral liquid formulations of metoprolol tartrate that are chemically and physically stable, pleasingly palatable, and have an acceptable shelf-life and in-use stability. The present disclosure offers technical advancement and has economic significance for elderly, and the needful patient community. The formulations of the present disclosure have a shelf-life of at least 18 months, 24 months, 30 months, or 36 months.

### SUMMARY

The present disclosure relates to stable oral liquid pharmaceutical formulations comprising metoprolol or pharmaceutically acceptable salts thereof, one or more preservatives, one or more cosolvents, and water. The present disclosure further relates to stable oral liquid pharmaceutical formulations comprising metoprolol or pharmaceutically acceptable salts thereof, one or more preservatives, one or more chelating agents, one or more sweeteners, and water. The formulations of the present disclosure are palatable, alcohol-free, and sugar-free. The present disclosure further relates to method of treatment of angina, hypertension, and myocardial infarction comprising administering to the subject in need thereof stable oral liquid pharmaceutical formulation comprising metoprolol or pharmaceutically acceptable salts thereof, one or more preservatives, one or more chelating agents, one or more sweeteners, and water.

In certain embodiments, the liquid pharmaceutical formulation comprises: (i) metoprolol or a pharmaceutically acceptable salt thereof; (ii) a preservative; (iii) a cosolvent; and (iv) water; wherein the pH of the formulation is about 2.5 to about 5.5.

In certain embodiments, the metoprolol is metoprolol tartrate. In certain embodiments, the metoprolol is about 0.1% w/v to about 10% w/v of the formulation. In certain embodiments, the metoprolol is about 0.5% w/v to about 7% w/v of the formulation. In certain embodiments, the metoprolol is about 0.75% w/v to about 5% w/v of the formulation. In certain embodiments, the metoprolol is about 0.75% w/v to about 3% w/v of the formulation.

In certain embodiments, the preservative comprises sodium benzoate, methyl paraben, propylparaben, ethyl paraben, butyl paraben, sorbic acid, potassium sorbate, sodium sorbate, or a combination thereof.

In certain embodiments, the preservative is about 0.01% w/v to about 15% w/v of the formulation. In certain embodiments, the preservative is about 0.02% w/v to about 10% w/v of the formulation. In certain embodiments, the preservative is about 0.05% w/v to about 5% w/v of the formulation.

In certain embodiments, the preservative comprises sodium benzoate. In certain embodiments, the sodium benzoate is about 0.01% w/v to about 15% w/v of the formulation. In certain embodiments, the sodium benzoate is about 0.02% w/v to about 10% w/v of the formulation. In certain embodiments, the sodium benzoate is about 0.05% w/v to about 5% w/v of the formulation.

In certain embodiments, the cosolvent comprises propylene glycol, glycerine, or combinations thereof. In certain embodiments, the cosolvent is about 1% w/v to about 50% w/v of the formulation. In certain embodiments, the cosolvent is about 2% w/v to about 40% w/v of the formulation. In certain embodiments, the cosolvent is about 3% w/v to about 30% w/v of the formulation.

In certain embodiments, the cosolvent comprises glycerine. In certain embodiments, the glycerine is about 1% w/v to about 50% w/v of the formulation. In certain embodiments, the glycerine is about 2% w/v to about 40% w/v of the formulation. In certain embodiments, the glycerine is about 3% w/v to about 30% w/v of the formulation.

In certain embodiments, the formulation further comprises a chelating agent. In certain embodiments, the chelating agent comprises disodium edetate, tetrasodium edetate, dipotassium edetate, calcium disodium edetate, L-cysteine, N-acetylL-cysteine, or a combination thereof.

In certain embodiments, the chelating agent is about 0.001% w/v to about 10% w/v of the formulation. In certain embodiments, the chelating agent is about 0.005% w/v to about 5% w/v of the formulation. In certain embodiments, the chelating agent is about 0.01% w/v to about 2% w/v of the formulation.

In certain embodiments, the chelating agent comprises disodium edetate. In certain embodiments, the disodium edetate is about 0.001% w/v to about 10% w/v of the formulation. In certain embodiments, the disodium edetate is about 0.005% w/v to about 5% w/v of the formulation. In certain embodiments, the disodium edetate is about 0.01% w/v to about 2% w/v of the formulation.

In certain embodiments, the formulation further comprises a sweetener. In certain embodiments, the sweetener comprises sucralose, acesulfame potassium, sodium saccharin, or a combination thereof. In certain embodiments, the sweetener is about 0.01% w/v to about 15% w/v of the formulation. In certain embodiments, the sweetener is about 0.02% w/v to about 10% w/v of the formulation. In certain embodiments, the sweetener is about 0.05% w/v to about 8% w/v of the formulation.

In certain embodiments, the formulation is stable for greater than 12 months. In certain embodiments, the formulation is stable for greater than 18 months.

In certain embodiments, the formulation does not comprise a buffer.

In certain embodiments, the liquid pharmaceutical formulation comprises: (i) about 0.1% w/v to about 10% w/v metoprolol tartrate; (ii) about 0.01% w/v to about 15% w/v preservative; (iii) about 1% w/v to about 50% w/v cosolvent; and (iv) water; wherein the pH of the formulation is about 2.5 to about 5.5.

In certain embodiments, the liquid pharmaceutical formulation comprises: (i) about 0.1% w/v to about 10% w/v metoprolol tartrate; (ii) about 0.01% w/v to about 15% w/v sodium benzoate; (iii) about 1% w/v to about 50% w/v glycerine; and (iv) water; wherein the pH of the formulation is about 2.5 to about 5.5.

In certain embodiments, the liquid pharmaceutical formulation comprises: (i) about 0.1% w/v to about 10% w/v metoprolol tartrate; (ii) about 0.01% w/v to about 15% w/v preservative; (iii) about 0.001% w/v to about 10% w/v chelating agent; (iv) about 0.01% w/v to about 15% w/v sweetener; (v) about 1% w/v to about 50% w/v cosolvent; and (v) water; wherein the pH of the formulation is about 2.5 to about 5.5.

In certain embodiments, the liquid pharmaceutical formulation comprises: (i) about 0.1% w/v to about 10% w/v metoprolol tartrate; (iii) about 0.01% w/v to about 15% w/v sodium benzoate; (ii) about 0.001% w/v to about 10% w/v disodium edetate; (iv) about 0.01% w/v to about 15% w/v sucralose; (v) about 1% w/v to about 50% w/v; and (v) water; wherein the pH of the formulation is about 2.5 to about 5.5.

In certain embodiments, the liquid pharmaceutical formulation comprises: (i) about 0.1% w/v to about 10% w/v metoprolol tartrate; (ii) about 0.01% w/v to about 15% w/v preservative; (iii) about 0.001% w/v to about 10% w/v chelating agent; (iv) about 0.01% w/v to about 15% w/v sweetener; (v) about 0.001% w/v to about 10% w/v thickening agent; (vi) about 0.001% w/v to about 5% w/v pH adjuster; (vii) about 1% w/v to about 50% w/v cosolvent; and (viii) water; wherein the formulation does not comprise a buffer, and wherein the pH of the formulation is about 2.5 to about 5.5.

In certain embodiments, the liquid pharmaceutical formulation comprises: (i) about 0.1% w/v to about 10% w/v metoprolol tartrate; (ii) about 0.01% w/v to about 15% w/v sodium benzoate; (iii) about 0.001% w/v to about 10% w/v disodium edetate; (iv) about 0.01% w/v to about 15% w/v sucralose; (v) about 0.001% w/v to about 10% w/v xanthan gum; (vi) about 0.001% w/v to about 5% w/v citric acid monohydrate; (vii) about 1% w/v to about 50% w/v glycerine; and (viii) water; wherein the formulation does not comprise a buffer, and wherein the pH of the formulation is about 2.5 to about 5.5.

In certain embodiments, the formulation is stable for at least 12 months. In certain embodiments, the formulation is stable for at least 18 months. In certain embodiments, the formulation is stable for at least 24 months. In certain embodiments, the formulation is stable for at least 30 months. In certain embodiments, the formulation is stable for at least 36 months.

In certain embodiments, the formulation does not have more than about 5% w/w of total degradation products at the end of shelf life.

In certain embodiments, the formulation does not have more than about 0.5% w/w of specified impurity Metoprolol Related Compound C at the end of shelf life.

In certain embodiments, the shelf life of the formulation is 12 months, 18 months, 24 months, 30 months or 36 months.

In certain embodiments, the present disclosure relates to a method of preparing stable liquid oral formulations comprising: (i) heating purified water in a first vessel to 65-85° C. and adding chelating agent while stirring until a clear solution is formed, and allowing the solution to cool to 25-35° C.; (ii) heating purified water in a second vessel to 40 C° to 45° C. and adding thickening agent while stirring until a clear solution is formed, and allowing the solution to cool to 25-35° C.; (iii) adding cosolvent to the solution of (i) under stirring and mixing for 10 to 15 minutes or until clear solution is formed; (iv) adding preservative to purified water in a third vessel under stirring, mixing for 10 to 15 minutes or until clear solution is formed and adding the solution to the solution of (i) under stirring and mixing for 10 to 15 minutes or until clear solution is formed; (v) adding metoprolol tartrate to purified water in a fourth vessel under stirring and mixing for 10 to 15 minutes or until clear solution is formed; (vi) adding the solution of (v) to the solution of (i) to which the solution of (ii) and (iv) has already been added under stirring and mixing for 10 to 15 minutes or until clear solution is formed; (vii) adding sweetener to water in a fifth vessel under stirring and mixing for 10 to 15 minutes or until clear solution is formed; (viii) adding the solution of (vii) to the solution of (i) to which the solutions of (ii), (iv) and (v) have been added, under stirring and mixing for 10 to 15 minutes or until clear solution is formed; (ix) adding pH adjuster to purified water in a sixth vessel under stirring and mixing for 5-10 minutes or until clear solution is formed; (x) adding the solution of (ix) to the solution of (i) to which the solutions of (ii), (iv), (v), and (vii) have been added and adjusting the pH of the solution to be between 3.8 to 4.2 with the solution of (ix) as needed; and (xi) adjusting the volume of the solution of (x) using purified water in quantity sufficient to make up the volume to 100%.

In certain embodiments, the present disclosure relates to a method of treating a disease or condition responsive to beta-blockers in a subject in need thereof, the method comprising administering a liquid pharmaceutical formulation comprising: (i) metoprolol or a pharmaceutically acceptable salt thereof; (ii) a preservative; (iii) a cosolvent; and (iv) water; wherein the formulation does not comprise a buffer, and wherein the pH of the formulation is about 2.5 to about 5.5.

In certain embodiments, the disease or condition responsive to beta-blockers comprises angina, hypertension, or myocardial infarction.

### DETAILED DESCRIPTION

Before the present compounds, formulations, and methods, among others, are described, it is to be understood that the inventions described and claimed herein are not limited to the particular processes, formulations, or methodologies described, as these may vary. It is also to be understood that the terminology used in the description is for the purpose of describing the particular versions or embodiments only and is not intended to limit the scope of the present inventions, which will be limited only by the appended claims. Unless defined otherwise, all technical and scientific terms used herein have the same meaning as commonly understood by one of ordinary skill in the art. Although any methods and materials similar or equivalent to those described herein can be used in the practice or testing of embodiments of the present invention, the preferred methods, devices, and materials are now described. All patents, patent applications, and other publications cited or otherwise mentioned herein are incorporated by reference in their entirety. Nothing herein is to be construed as an admission that the inventions as recited in the appended claims are not entitled to antedate such disclosure(s) by virtue of prior invention.

As used herein and in the appended claims, the use of “a,” “an,” and/or “the” is intended to include both the singular and plural (e.g., “one or more”) unless the context clearly dictates otherwise. Thus, for example, reference to “an excipient” is a reference to one or more excipients and equivalents thereof known to those skilled in the art, and so forth. The term “about” is used to indicate that a value includes the standard level of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or”. The terms “comprise”, “have”, and “include” are open-ended linking verbs. Any forms or tenses of one or more of these verbs “comprises,” “comprising,” “has,” “having,” “includes,” and “including” are also open-ended. For example, any method that “comprises,” “has” or “includes” one or more steps is not limited to possessing only those one or more steps and also covers other unlisted steps. Unless specified, “%” may refer to a percent by weight percent, or a percent by volume, or a percent weight by unit volume, and the relevant units would be immediately apparent to one of ordinary skill in the art based on the context.

The term “pharmaceutically acceptable” means a compound or ingredient that is compatible with the other ingredients in a pharmaceutical formulation and not injurious to an intended subject when administered in normal or therapeutically effective amounts. As used herein, an “intended subject” includes animals and/or humans. The terms “patient” and “subject” may be used interchangeably. The term “stable” as used herein refers to metoprolol tartrate oral liquid formulations that do not have more than about 5% w/w of total degradation products at the end of a given storage period. Stability is assessed by High Performance Fluid Chromatography (HPLC) or any other known testing method. The term “total degradation products” as used herein refers to the total impurities in the formulations of the present disclosure. The term “shelf life” indicates the period over which the formulations of the present disclosure remain stable at a given storage condition.

The present disclosure relates to stable oral liquid pharmaceutical formulations comprising metoprolol or pharmaceutically acceptable salts thereof, one or more preservatives, one or more cosolvents, and water. The present disclosure further relates to stable oral liquid pharmaceutical formulations comprising metoprolol or pharmaceutically acceptable salts thereof, one or more preservatives, one or more chelating agents, one or more sweeteners, and water. The present disclosure further relates to method of treatment of angina, hypertension, and myocardial infarction comprising administering to the subject in need thereof stable oral liquid pharmaceutical formulation comprising metoprolol or pharmaceutically acceptable salts thereof, one or more preservatives, one or more chelating agents, one or more sweeteners, and water.

In one embodiment, the stable oral liquid formulations of metoprolol tartrate that are chemically and physically stable, pleasingly palatable, have an acceptable shelf-life and in-use stability. In another embodiment, the formulations of the present disclosure have a shelf-life of at least 18 months, 24 months, 30 months, or 36 months.

In some embodiments, the dosage form of the present disclosure is a stable, oral liquid dosage form. A liquid dosage can be advantageous as it can be suitably adjusted to meet the dosing needs of the patients. Such a liquid dosage form can provide significant benefits over the solid tablet dosage forms where titration is required. Liquid dosage forms allow for more precise and accurate dose adjustment. Liquid dosage forms are particularly useful with elderly patients, or patients suffering from dysphagia. Liquid dosage forms can also be useful in the case of severely ill or incapacitated patients needing medications administered through gastric or nasal tubes.

In one embodiment, the formulations of the present disclosure comprise the active agent metoprolol or pharmaceutically acceptable salts thereof. In a further embodiment, the formulations of the present disclosure comprise metoprolol tartrate. In another embodiment, the formulation of the present disclosure comprises any known polymorphic form of metoprolol tartrate. In one embodiment, the stable oral liquid formulation of the present disclosure comprises about 0.1% w/v to about 10% w/v of metoprolol tartrate. “w/v” is expressed as the mass concentration of the solute in solution. In some embodiments, the stable oral liquid formulation of the present disclosure comprises from about 0.5% w/v to about 7% w/v of metoprolol tartrate. In some embodiments, the stable oral liquid formulation of the present disclosure comprises from about 0.75% w/v to about 5% w/v of metoprolol tartrate. In some embodiments, the stable oral liquid formulation of the present disclosure comprises from about 0.75% w/v to about 3% w/v of metoprolol tartrate. The stable oral liquid formulation of the present disclosure can comprise from about 0.1% w/v to about 10% w/v metoprolol tartrate, e.g., from 0.5% w/v to 7% w/v metoprolol tartrate, from 0.75% w/v to 5% w/v metoprolol tartrate, or from 0.75% w/v to 3% w/v of metoprolol tartrate. In terms of upper limits, the stable oral liquid formulation comprises less than about 10% w/v metoprolol tartrate, e.g., less than 7% w/v, less than 5% w/v, or less than 3% w/v. In terms of lower limits, the stable oral liquid formulation comprises more than about 0.1% w/v metoprolol tartrate, e.g., more than 0.5% w/v more than 1% w/v. In some embodiments, the formulations of the present disclosure comprise about 1% w/v metoprolol tartrate. In one embodiment, the formulations of the present disclosure comprise therapeutically effective amount of metoprolol tartrate.

In some embodiments, the formulation of the present disclosure comprises water. In some embodiments, water is a liquid carrier or solvent of the formulation. In some embodiments, the formulation comprises water in quantity sufficient to make up the volume to 100%. Examples disclosed herein include purified water.

In another embodiment, the formulation of the present disclosure comprises at least one cosolvent. Cosolvents may include, e.g., propylene glycol, glycerine, polyethylene glycol, alcohol, and combinations thereof. In some embodiments, the formulation of the present disclosure comprises at least one cosolvent selected from, but not limited to, propylene glycol, glycerine, and combinations thereof.

In one embodiment, the formulation of the present disclosure comprises about 1% w/v to about 50% w/v cosolvent. The stable oral liquid formulation of the present disclosure can comprise one or more cosolvents (other than water) in the amount of from about 1% w/v to about 50% w/v cosolvent, e.g., from 2% w/v to 40% w/v cosolvent, from 3% w/v to 30% w/v cosolvent, from 3% w/v to 25% w/v cosolvent, or from 5% w/v to 20% w/v cosolvent. In some embodiments, the formulation of the present disclosure comprises about 2% w/v to about 40% w/v cosolvent. In some embodiments, the formulation of the present disclosure comprises about 3% w/v to about 30% w/v cosolvent. In some embodiments, the formulation of the present disclosure comprises about 3% w/v to about 25% w/v cosolvent. In some embodiments, the formulation of the present disclosure comprises about 5% w/v to about 20% w/v cosolvent. In terms of upper limits, the formulation comprises less than about 50% w/v cosolvent, e.g., less than 40% w/v, less than 30% w/v, less than 25% w/v, or less than 20% w/v. In terms of lower limits, the formulation comprises more than about 1% w/v cosolvent, e.g., more than 2% w/v, more than 3% w/v, or more than 5% w/v. In one embodiment, the formulation of the present disclosure comprises about 10% w/v cosolvent.

In some embodiments, the cosolvent is glycerine. In one embodiment, the formulation of the present disclosure comprises about 1% w/v to about 50% w/v glycerine. In some embodiments, the formulation of the present disclosure comprises about 2% w/v to about 40% w/v glycerine. In some embodiments, the formulation of the present disclosure comprises about 3% w/v to about 30% w/v glycerine. In some embodiments, the formulation of the present disclosure comprises about 3% w/v to about 25% w/v glycerine. In some embodiments, the formulation of the present disclosure comprises about 5% w/v to about 20% w/v glycerine. In terms of upper limits, the formulation comprises less than about 50% w/v glycerine, e.g., less than about 40% w/v, less than about 30% w/v, less than about 25% w/v, or less than about 20% w/v. In terms of lower limits, the formulation comprises more than about 1% w/v glycerine, e.g., more than about 2% w/v, more than about 3% w/v, or more than about 5% w/v. In one embodiment, the formulation of the present disclosure comprises about 10% w/v glycerine.

In one embodiment, the formulations of the present disclosure comprise at least one chelating agent. Chelating agents may include, e.g., disodium edetate, tetrasodium edetate, dipotassium edetate, calcium disodium edetate, L-cysteine, N-acetyl-L-cysteine, and combinations thereof. In a further embodiment, the formulation of the present disclosure comprises a chelating agent such as but not limited to disodium edetate, tetrasodium edetate, dipotassium edetate, calcium disodium edetate, L-cysteine, N-acetyl-L-cysteine, and combinations thereof. In yet another embodiment, the formulation of the present disclosure comprises a chelating agent disodium edetate. In another embodiment, the formulations of the present disclosure comprise about 0.001% w/v to about 10% w/v chelating agent. In still another embodiment, the formulations of the present disclosure comprise about 0.005% w/v to about 5% w/v chelating agent. In a further embodiment, the formulations of the present disclosure comprise about 0.01% w/v to about 2% w/v chelating agent. In some embodiments, the formulations of the present disclosure comprise about 0.02% w/v to about 1% w/v chelating agent. In terms of upper limits, the formulation comprises less than about 10% w/v chelating agent, e.g., less than 8% w/v, less than 5% w/v, less than 2% w/v, or less than 1% w/v. In terms of lower limits, the formulation comprises more than about 0.001% w/v chelating agent, e.g., more than 0.005% w/v, more than 0.0075% w/v, or more than 0.01% w/v. In one embodiment, the formulations of the present disclosure comprise about 0.05% w/v chelating agent.

In a further embodiment, the formulations of the present disclosure comprise at least one preservative. Preservatives may include, e.g., sodium benzoate, methyl paraben, propylparaben, ethyl paraben, butyl paraben, sorbic acid, potassium sorbate, sodium sorbate, and combinations thereof. In one embodiment, the formulation of the present disclosure comprises preservatives such as but not limited to sodium benzoate, methyl paraben, propylparaben, ethyl paraben, butyl paraben, sorbic acid, potassium sorbate, sodium sorbate, or combinations thereof. In another embodiment, the formulations of the present disclosure comprise about 0.01% w/v to about 15% w/v preservative. In yet another embodiment, the formulations of the present disclosure comprise about 0.02% w/v to about 10% w/v preservative. In a further embodiment, the formulations of the present disclosure comprise about 0.05% w/v to about 5% w/v preservative. In another embodiment, the formulations of the present disclosure comprise about 0.075% w/v to about 3% w/v preservative. In terms of upper limits, the formulation comprises less than about 15% w/v preservative, e.g., less than 10% w/v, less than 5% w/v, less than 3% w/v, or less than 1% w/v. In terms of lower limits, the formulation comprises more than about 0.01% w/v chelating agent, e.g., more than 0.02% w/v, more than 0.05% w/v, or more than 0.075% w/v. In one embodiment, the formulation of the present disclosure comprises 0.2% w/v preservative.

In one embodiment, the formulations of the present disclosure comprise at least one sweetener. Sweeteners may include, e.g., sucralose, acesulfame potassium, sodium saccharin, and combinations thereof. In a further embodiment, the formulation of the present disclosure comprises sweetener such as but not limited to sucralose, acesulfame potassium, sodium saccharin or combinations thereof. In another embodiment, the formulations of the present disclosure comprise about 0.01% w/v to about 15% w/v sweetener. In still another embodiment, the formulations of the present disclosure comprise about 0.02% w/v to about 10% w/v sweetener. In a further embodiment, the formulations of the present disclosure comprise about 0.05% w/v to about 8% w/v sweetener. In another embodiment, the formulations of the present disclosure comprise about 0.05% w/v to about 5% w/v sweetener. In terms of upper limits, the formulation comprises less than about 15% w/v sweetener, e.g., less than 10% w/v, less than 8% w/v, or less than 5% w/v. In terms of lower limits, the formulation comprises more than about 0.01% w/v sweetener, e.g., more than 0.02% w/v, or more than 0.05% w/v.

In one embodiment, the formulations of the present disclosure comprise at least one thickening agent. Thickening agents may include, e.g., xanthan gum, sodium carboxymethyl cellulose, co-processed sodium carboxymethyl cellulose and microcrystalline cellulose, and combinations thereof. In one embodiment, the formulation of the present disclosure comprises the thickening agent xanthan gum. In another embodiment, the thickening agent is present in the formulations of the present disclosure at about 0.001% w/v to about 10% w/v. In yet another embodiment, the thickening agent is present in the formulations of the present disclosure at about 0.005% w/v to about 8% w/v. In one embodiment, the thickening agent is present in the formulations of the present disclosure at about 0.0075% w/v to about 5% w/v. In a further embodiment, the thickening agent is present in the formulations of the present disclosure at about 0.01% w/v to about 3% w/v. In some embodiments, the thickening agent is present in the formulations of the present disclosure at about 0.05% w/v to about 2% w/v. In terms of upper limits, the formulation comprises less than about 10% w/v thickening agent, e.g., less than 10% w/v, less than 8% w/v, less than 5% w/v, less than 3% w/v or less than 2% w/v. In terms of lower limits, the formulation comprises more than about 0.001% w/v thickening agent, e.g., more than 0.001% w/v, more than 0.005% w/v, more than 0.0075% w/v, more than 0.01% w/v, or more than 0.05% w/v.

In a further embodiment, the formulations of the present disclosure comprise at least one pH adjuster, e.g., citric acid monohydrate, hydrochloric acid, ascorbic acid, lactic acid, succinic acid and combinations thereof. In one embodiment, the formulation of the present disclosure comprises the pH adjuster citric acid monohydrate. In another embodiment, the pH adjuster is present in the formulations of the present disclosure at about 0.001% w/v to about 5% w/v. In yet another embodiment, the pH adjuster is present in the formulations of the present disclosure at about 0.005% w/v to about 4% w/v. In a further embodiment, the pH adjuster is present in the formulations of the present disclosure at about 0.01% w/v to about 3% w/v. In some embodiments, the pH adjuster is present in the formulations of the present disclosure at about 0.05% w/v to about 2% w/v. In terms of upper limits, the formulation comprises less than about 5% w/v pH adjuster, e.g., less than 5% w/v, less than 4% w/v, less than 3% w/v, or less than 2% w/v. In terms of lower limits, the formulation comprises more than about 0.001% w/v pH adjuster, e.g., more than 0.001% w/v, more than 0.005% w/v, more than 0.01% w/v, or more than 0.05% w/v.

In another embodiment of the present disclosure, pH of the oral liquid formulation of metoprolol tartrate is in the range of about 2.5 to about 5.5. The pH adjuster additions alter the pH value of the aqueous formulation so that the pH value is in a range of from about 2.5 to about 5.5. The pH of the stable oral liquid formulation of metoprolol tartrate herein can, for example, range from about 2.5 to about 5.5, e.g., from 3.0 to 5.0, from 3.2 to 4.8, from 3.5 to 4.8, from 3.8 to 4.5, or from 3.8 to 4.2. In a further embodiment of the present disclosure, pH of the oral liquid formulation of metoprolol tartrate is in the range of about 3.0 to about 5.0. In one embodiment of the present disclosure, pH of the oral liquid formulation of metoprolol tartrate is in the range of about 3.2 to about 4.8. In another embodiment of the present disclosure, pH of the oral liquid formulation of metoprolol tartrate is in the range of about 3.5 to about 4.8. In a further embodiment of the present disclosure, pH of the oral liquid formulation of metoprolol tartrate is in the range of about 3.8 to about 4.5. In another embodiment of the present disclosure, pH of the oral liquid formulation of metoprolol tartrate is in the range of about 3.8 to about 4.2. In terms of upper limits, the pH adjuster additions alter the pH value of the aqueous formulation so that the pH value can be less than 5.5, e.g., less than 5.0, less than 4.8, less than 4.5, or less than 4.2. In terms of lower limits, the pH adjuster additions alter the pH value of the aqueous formulation so that the pH value can be greater than 2.5, e.g., greater than 3.0, greater than 3.2, greater than 3.5, or greater than 3.8.

In one embodiment, the formulations of the present disclosure are buffer-free. The present disclosure demonstrates that oral liquid formulations within the claimed pH ranges provide for stable oral liquid formulations of metoprolol tartrate. That buffer-free formulations can demonstrate increased stability and shelf-life was unexpected.

In a further embodiment, the formulations of the present disclosure may optionally comprise a flavouring agent. In another embodiment, the formulations of the present disclosure may optionally comprise a colouring agent.

In one embodiment, the formulation of the present disclosure is in the form of solution for oral administration. In one embodiment, the formulation of the present disclosure is in the form of syrup for oral administration.

In one embodiment of the present disclosure stable oral liquid pharmaceutical formulations comprising metoprolol or pharmaceutically acceptable salts thereof, one or more preservatives, one or more chelating agents, one or more sweeteners, and water are buffer-free.

Some or all of these components may be considered optional. In some cases, the disclosed compositions may expressly exclude one or more of the aforementioned components, e.g., via claim language. For example, claim language may be modified to recite that the disclosed compositions, methods, etc., do not utilize or comprise one or more of the aforementioned additives.

In one embodiment, the stable oral liquid formulation of the present disclosure comprises about 0.1% w/v to about 10% w/v metoprolol tartrate, about 0.001% w/v to about 10% w/v chelating agent, about 0.01% w/v to about 15% w/v preservative, 0.01% w/v to about 15% w/v sweetener and water. In another embodiment, the stable oral liquid formulation of the present disclosure comprises about 0.1% w/v to about 10% w/v metoprolol tartrate, about 0.001% w/v to about 10% w/v chelating agent, about 0.01% w/v to about 15% w/v preservative, 0.01% w/v to about 15% w/v sweetener and water in quantity sufficient to make up the volume to 100%. In another embodiment, the stable oral liquid formulation of the present disclosure comprises about 0.1% w/v to about 10% w/v metoprolol tartrate, about 1% w/v to about 50% w/v cosolvent, about 0.001% w/v to about 10% w/v chelating agent, about 0.01% w/v to about 15% w/v preservative, about 0.01% w/v to about 15% w/v sweetener, about 0.001% w/v to about 10% w/v thickening agent, about 0.001% w/v to about 5% w/v pH adjuster and water in quantity sufficient to make up the volume to 100%. In a further embodiment, the stable oral liquid formulation of the present disclosure comprises metoprolol tartrate, glycerine, disodium edetate, sodium benzoate, sucralose, xanthan gum, citric acid monohydrate and water. In one embodiment, the stable oral liquid formulation of the present disclosure comprises about 0.1% w/v to about 10% w/v metoprolol tartrate, about 0.001% w/v to about 10% w/v disodium edetate, about 0.01% w/v to about 15% w/v sodium benzoate, 0.01% w/v to about 15% w/v sucralose and water. In another embodiment, the stable oral liquid formulation of the present disclosure comprises about 0.1% w/v to about 10% w/v metoprolol tartrate, about 0.001% w/v to about 10% w/v disodium edetate, about 0.01% w/v to about 15% w/v sodium benzoate, 0.01% w/v to about 15% w/v sucralose and water in quantity sufficient to make up the volume to 100%. In another embodiment, the stable oral liquid formulation of the present disclosure comprises about 0.1% w/v to about 10% w/v metoprolol tartrate, about 1% w/v to about 50% w/v glycerine, about 0.001% w/v to about 10% w/v disodium edetate, about 0.01% w/v to about 15% w/v sodium benzoate, 0.01% w/v to about 15% w/v sucralose, about 0.001% w/v to about 10% w/v xanthan gum, about 0.001% w/v to about 5% w/v citric acid monohydrate and water in quantity sufficient to make up the volume to 100%.

The present disclosure provides for metoprolol tartrate oral liquid formulations with acceptable shelf-life. In one embodiment, the formulations of the present disclosure have a shelf-life of at least 18 months. In another embodiment, the formulations of the present disclosure have a shelf-life of at least 2 years or 24 months. In a further embodiment, the formulations of the present disclosure have a shelf-life of at least 30 months. In yet another embodiment, the formulations of the present disclosure have a shelf-life of at least 3 years or 36 months.

In one embodiment, the oral liquid formulations of the present disclosure are stable at 25° C.±2° C./60%±5% RH for at least 18 months. In another embodiment, the oral liquid formulations of the present disclosure are stable at 25° C.±2° C./60%±5% RH for at least 2 years or 24 months. In a further embodiment, the formulations of the present disclosure are stable at 5 25° C.±2° C./60%±5% RH for 2 years or 24 months. In one embodiment, the formulations of the present disclosure are stable at 25° C.±2° C./60%±5% RH for 30 months. In yet another embodiment, the formulations of the present disclosure are stable at 25° C.±2° C./60%±5% RH for 36 months. In a further embodiment, the formulations of the present disclosure are stable at 40° C.±2° C./75%±5% RH for 6 months.

In some embodiments, the stable oral liquid formulations of metoprolol tartrate have about 5% w/w, about 4% w/w, about 3% w/w, about 2.5% w/w, about 2% w/w, about 1.5% w/w, or about 1% w/w total degradation products. In some embodiments, the stable oral liquid formulations of metoprolol tartrate have about 5% w/w total degradation products. In some embodiments, the stable oral liquid formulations of metoprolol tartrate have about 4% w/w total degradation products. In some embodiments, the stable oral liquid formulations of metoprolol tartrate have about 3% w/w total degradation products. In some embodiments, the stable oral liquid formulations of metoprolol tartrate have about 2.5% w/w total degradation products. In some embodiments, the stable oral liquid formulations of metoprolol tartrate have about 2% w/w total degradation products. In some embodiments, the stable oral liquid formulations of metoprolol tartrate have about 1.5% w/w total degradation products. In some embodiments, the stable oral liquid formulations of metoprolol tartrate have about 1% w/w total degradation products.

In some embodiments, the stable oral liquid formulations of metoprolol tartrate have not more than about 0.5% w/w, about 0.4% w/w, about 0.3% w/w, about 0.25% w/w, or about 0.2% w/w specified impurity Metoprolol Related Compound C. In some embodiments, the stable oral liquid formulations of metoprolol tartrate have not more than about 0.5% w/w Metoprolol Related Compound C. In some embodiments, the stable oral liquid formulations of metoprolol tartrate have not more than about 0.4% w/w Metoprolol Related Compound C. In some embodiments, the stable oral liquid formulations of metoprolol tartrate have not more than about 0.3% w/w Metoprolol Related Compound C. In some embodiments, the stable oral liquid formulations of metoprolol tartrate have not more than about 0.25% w/w Metoprolol Related Compound C. In some embodiments, the stable oral liquid formulations of metoprolol tartrate have not more than about 0.2% w/w Metoprolol Related Compound C.

In some embodiments, the given storage period is about 6 months, about 9 months, about 12 months, about 18 months, about 24 months, about 30 months or about 36 months at temperature and humidity condition of 25° C.±2° C. and 60%±5% RH. In some embodiments, the given storage period is about 6 months at temperature and humidity condition of 40° C.±2° C. and 75%±5% RH.

In some embodiments, the stable oral liquid formulations of metoprolol tartrate are physically and chemically stable. In some embodiments, stable oral liquid formulations of the present disclosure have a shelf life at ambient temperature of equal to or greater than 18 months, 24 months, 30 months, or 36 months. In some embodiments, stable oral liquid formulations of the present disclosure have a shelf life at about 25° C. of equal to or greater than 18 months, 24 months, 30 months, or 36 months. In some embodiments, the formulations do not have more than about 5% w/w of total degradation products even after being stored in liquid form for an extended period of time.

In one embodiment, the liquid formulations of the present disclosure are homogeneous. Mixing methods may be employed in the process of preparation of the formulations of the present disclosure. Mixing methods encompass any type of mixing that results in a homogeneous oral liquid formulation.

In one embodiment, the present disclosure provides a method of preparing stable oral formulations of the present disclosure comprising
(i) heating purified water in a vessel to 65-85° C. and adding the required quantity of chelating agent under stirring, mixing for 10 to 15 minutes or until clear solution is formed, and allowing the solution to cool to 25-35° C.;
(ii) heating purified water in a separate vessel to 40 C° to 45° C. and adding thickening agent under stirring, mixing for 10 to 15 minutes or until clear solution is formed, allowing the solution to cool to 25-35° C. and adding to the solution of (i);
(iii) adding cosolvent to the solution of (i) under stirring and mixing for 10 to 15 minutes or until clear solution is formed;
(iv) adding preservative to purified water in a separate vessel under stirring, mixing for 10 to 15 minutes or until clear solution is formed and adding the solution to the solution of (i) under stirring and mixing for 10 to 15 minutes or until clear solution is formed;
(v) adding metoprolol tartrate to purified water in another separate vessel under stirring and mixing for 10 to 15 minutes or until clear solution is formed;
(vi) adding the solution of (v) to the solution of (i) to which the solution of (ii) and (iv) has already been added under stirring and mixing for 10 to 15 minutes or until clear solution is formed;
(vii) adding sweetener to water in another separate vessel under stirring and mixing for 10 to 15 minutes or until clear solution is formed;
(viii) adding the solution of (vii) to the solution of (i) to which the solutions of (ii), (iv) and (v) have been added, under stirring and mixing for 10 to 15 minutes or until clear solution is formed;
(ix) adding pH adjuster to purified water in another separate vessel under stirring and mixing for 5-10 minutes or until clear solution is formed;
(x) adding the solution of (ix) to the solution of (i) to which the solutions of (ii), (iv), (v), and (vii) have been added and adjusting the pH of the solution to be between 3.8 to 4.2 with the solution of (ix) as needed; and
(xi) adjusting the volume of the solution of (x) using purified water in quantity sufficient to make up the volume to 100%.

For clarity, the disclosure provides that any of (ii), (iii), or (iv) can be added to (i) in any order. Thus, e.g., the solution of (v) can be added to the solution of (i), to which the solution of (ii) and (iv) have been added, independently of whether (iii) has been added.

In another embodiment, the present disclosure provides method of preparing stable oral formulations of the present disclosure comprising
(i) heating purified water in a vessel to 65-85° C. and adding required quantity of disodium edetate under stirring, mixing for 10 to 15 minutes or until clear solution is formed, and allowing the solution to cool to 25-35° C.;
(ii) heating purified water in a separate vessel to 40 C° to 45° C. and adding xanthan gum under stirring, mixing for 10 to 15 minutes or until clear solution is formed, and allowing the solution to cool to 25-35° C.;
(iii) adding glycerine to the solution of (i) under stirring and mixing for 10 to 15 minutes or until clear solution is formed;
(iv) adding sodium benzoate to purified water in a separate vessel under stirring, mixing for 10 to 15 minutes or until clear solution is formed and adding the solution to the solution of (i) under stirring and mixing for 10 to 15 minutes or until clear solution is formed;
(v) adding metoprolol tartrate added to purified water in another separate vessel under stirring and mixing for 10 to 15 minutes or until clear solution is formed;
(vi) adding the solution of (v) to the solution of (i) to which the solution of (ii) and (iv) has already been added, under stirring and mixing for 10 to 15 minutes or until clear solution is formed;
(vii) adding sucralose to purified water in another separate vessel under stirring and mixing for 10 to 15 minutes or until clear solution is formed;
(viii) adding the solution of (vii) to the solution of (i) to which the solutions of (ii), (iv) and (v) have been added, under stirring and mixing for 10 to 15 minutes or until clear solution is formed;
(ix) adding citric acid monohydrate to purified water in another separate vessel under stirring and mixing for 5-10 minutes or until clear solution is formed;
(x) adding the solution of (ix) to the solution of (i) to which the solutions of (ii), (iv), (v), and (vii) have been added and adjusting the pH of the solution to 3.8 to 4.2 with the solution of (ix) as needed; and
(xi) adjusting the volume of the solution of (x) using purified water in quantity sufficient to make up the volume to 100%.

For clarity, the disclosure provides that any of (ii), (iii), or (iv) can be added to (i) in any order. Thus, e.g., the solution of (v) can be added to the solution of (i), to which the solution of (ii) and (iv) have been added, independently of whether (iii) has been added.

In a further embodiment, the formulations of the present disclosure are for oral administration. In another embodiment, the formulations of the present disclosure are provided in the form of a kit comprising the formulation and an administration device. In one embodiment, the administration device provided in the kit of the present disclosure includes, but is not limited to, syringe, dosing cup or the like. In a further embodiment, the present disclosure relates to a pharmaceutical kit comprising stable oral liquid formulation of metoprolol tartrate and at least one administration device. In some embodiments, optionally a set of instructions may be included in the kit.

In yet another embodiment, the present disclosure provides for method of administration of stable oral liquid formulation of metoprolol tartrate comprising withdrawing the prescribed volume of the stable oral liquid formulation of metoprolol tartrate using administration device and administering orally to the patient in need thereof the withdrawn volume of the formulation.

Some embodiments relate to method of treatment of a disease or condition that can be treated or prevented with metoprolol or pharmaceutically acceptable salts thereof comprising administering to the subject in need thereof stable oral liquid formulations of metoprolol or pharmaceutically acceptable salts thereof of the present disclosure. Some embodiments relate to method of treatment of a disease or condition that can be treated or prevented with beta-blockers comprising administering to the subject in need thereof stable oral liquid formulations of metoprolol of the present disclosure. One embodiment relates to method of treatment of angina, hypertension, and myocardial infarction comprising administering to the subject in need thereof stable oral liquid formulations of metoprolol of the present disclosure. The present disclosure relates to method of treatment of angina, hypertension, and myocardial infarction comprising administering to the subject in need thereof stable oral liquid pharmaceutical formulation comprising metoprolol or pharmaceutically acceptable salts thereof, one or more sweeteners, one or more preservatives, one or more chelating agents and water. Another embodiment relates to method of treatment of angina, hypertension, and myocardial infarction comprising administering to the subject in need thereof stable oral liquid formulations of metoprolol of the present disclosure. The present disclosure further relates to method of treatment of angina, hypertension, and myocardial infarction comprising administering to the subject in need thereof stable oral liquid pharmaceutical formulation comprising metoprolol or pharmaceutically acceptable salts thereof, one or more sweeteners, one or more preservatives, one or more chelating agents and water.

While reference to exemplary embodiments has been made herein, it will be understood by those skilled in the art that various changes may be made, and equivalents may be substituted for elements thereof without departing from the scope of the invention. In addition, many modifications may be made to adapt a particular situation or material to the teachings without departing from the essential scope thereof. Therefore, it is intended that the disclosure is not limited to the embodiments disclosed but include all embodiments falling within the scope thereof. These detailed descriptions, by way of the following examples, serve to exemplify the above general descriptions and embodiments which form part of the invention. These detailed descriptions are presented for illustrative purposes only and are not intended as a restriction on the scope of the invention.

### EXAMPLES

### Example 1. Metoprolol Tartrate Oral Solution 10 mg/mL (F1)

The formulation of Example 1 is detailed in Table 1.

TABLE 1
Metoprolol Tartrate Oral Formulation

| Sr. No. | Excipients | % w/v |
|---|---|---|
| 1 | Metoprolol tartrate* | 1.0 |
| 2 | Sodium benzoate | 0.20 |
| 3 | Xanthan gum | 0.10 |
| 4 | Glycerine | 10.0 |
| 5 | Sucralose | 0.10 |
| 6 | Disodium Edetate | 0.05 |
| 7 | Citric acid monohydrate ** | 0.20 |
| 8 | Purified water | q.s. to 100% |

*Calculate the Metoprolol tartrate quantity as per quantity required.
** Citric Acid Monohydrate (20% Solution) additional quantity may be required to get the pH in desired range.

The formulation of Example 1 was prepared according to the following procedure: (i) heating purified water in a vessel to 65-85° C. and adding the required quantity of disodium edetate under stirring, mixing for 10 to 15 minutes or until clear solution is formed, and allowing the solution to cool to 25-35° C.; (ii) heating purified water in a separate vessel to 40 C° to 45° C. and adding xanthan gum under stirring, mixing for 10 to 15 minutes or until clear solution is formed, and allowing the solution to cool to 25-35° C.; (iii) adding glycerine to the solution of (i) under stirring and mixing for 10 to 15 minutes or until clear solution is formed; (iv) adding sodium benzoate to purified water in a separate vessel under stirring, mixing for 10 to 15 minutes or until clear solution is formed and adding the solution to the solution of (i) under stirring and mixing for 10 to 15 minutes or until clear solution is formed; (v) adding metoprolol tartrate to purified water in another separate vessel under stirring and mixing for 10 to 15 minutes or until clear solution is formed; (vi) adding the solution of (v) to the solution of (i) to which the solution of (ii) and (iv) has been added under stirring and mixing for 10 to 15 minutes or until clear solution is formed; (vii) adding sucralose to purified water in another separate vessel under stirring and mixing for 10 to 15 minutes or until clear solution is formed; (viii) adding the solution of (vii) to the solution of (i) to which the solutions of (ii), (iv), and (v) have been added, under stirring and mixing for 10 to 15 minutes or until clear solution is formed; (ix) adding citric acid monohydrate to purified water in another separate vessel under stirring and mixing for 5-10 minutes or until clear solution is formed; (x) adding the solution of (ix) to the solution of (i) to which the solutions of (ii), (iv), (v), and (vii) have been added and adjusting the pH of the solution to 3.8 to 4.2 with the solution of (ix) as needed; and (xi) adjusting the volume of the solution of (x) using purified water in quantity sufficient to make up the volume to 100%.

The formulation described above was tested for chemical and physical stability at room temperature 25±2° C. and a relative humidity of 60±5% while the container comprising the formulation is stored in an upright position.

| | | | 3 | 6 | 9 | 12 |
|---|---|---|---|---|---|---|
| Test | Acceptance Criteria | Initial | Months | Months | Months | Months |
| Description | Clear, colorless | Complies | Complies | Complies | Complies | Complies |
| | solution, free from | | | | | |
| | any visible foreign | | | | | |
| | and particulate | | | | | |
| | matter, free of | | | | | |
| | precipitation and hazy | | | | | |
| | mass. | | | | | |

Organic Impurities (By HPLC)

| | | | | | | |
|---|---|---|---|---|---|---|
| Metoprolol | Not more than 0.2% | Below | Below | Below | Below | Below |
| Related | | LOQ | LOQ | LOQ | LOQ | LOQ |
| Compound | | (0.02%) | (0.02%) | (0.02%) | (0.02%) | (0.02%) |
| C | | | | | | |
| Total | Not more than 1.0% | Below | 0.1% | 0.1% | Below | Below |
| degradation | | LOQ | | | LOQ | LOQ |
| products | | | | | | |

Assay (By HPLC)

| | | | | | | |
|---|---|---|---|---|---|---|
| Assay | 90-110% | 103.2 | 100.3 | 101.7 | 100.9 | 100.8 |

The formulation remains physically and chemically stable at least 12 months at 25±2° C. and 60±5% RH.

The formulation described above was then tested for chemical and physical stability at room temperature 25±2° C. and a relative humidity (RH) of 60±5% while the container comprising the formulation is stored in horizontal position.

| | | | 3 | 6 | 9 | 12 |
|---|---|---|---|---|---|---|
| Test | Acceptance Criteria | Initial | Months | Months | Months | Months |
| Description | Clear, colorless | Complies | Complies | Complies | Complies | Complies |
| | solution, free from | | | | | |
| | any visible foreign | | | | | |
| | and particulate | | | | | |
| | matter, free of | | | | | |
| | precipitation and hazy | | | | | |
| | mass. | | | | | |

Organic Impurities (By HPLC)

| | | | | | | |
|---|---|---|---|---|---|---|
| Metoprolol | Not more than 0.2% | Below | Below | Below | Below | Below |
| Related | | LOQ | LOQ | LOQ | LOQ | LOQ |
| Compound | | (0.02%) | (0.02%) | (0.02%) | (0.02%) | (0.02%) |
| C | | | | | | |
| Total | Not more than 1.0% | Below | 0.1% | 0.1% | Below | Below |
| degradation | | LOQ | | | LOQ | LOQ |
| products | | | | | | |

Assay (By HPLC)

| | | | | | | |
|---|---|---|---|---|---|---|
| Assay | 90-110% | 103.2 | 101.5 | 101.6 | 101.0 | 100.9 |

The formulation remains physically and chemically stable at least 12 months at 25±2° C. and 60±5% RH.

The formulation is physically and chemically stable.

### Example 2. Metoprolol Tartrate Oral Solution 10 mg/mL (F2)

The formulation of Example 2 is detailed in Table 2.

TABLE 2
Metoprolol Tartrate Oral Formulation

| Sr. No. | Excipients | % w/v |
|---|---|---|
| 1 | Metoprolol tartrate* | 1.0 |
| 2 | Sodium benzoate | 0.20 |
| 3 | Xanthan gum | 0.10 |
| 4 | Glycerine | 10.0 |
| 5 | Sucralose | 0.10 |
| 6 | Disodium Edetate | 0.06 |
| 7 | Citric acid monohydrate ** | 0.20 |
| 8 | Purified water | q.s. to 100% |

*Calculate the Metoprolol tartrate quantity as per quantity required.
** Citric Acid Monohydrate (20% Solution) additional quantity may be required to get the pH in desired range.

Process of Preparation—Metoprolol Oral Solution was prepared by process as discussed in Example 1.

### Example 3. Metoprolol Tartrate Oral Solution 10 mg/mL (F3)

The formulation of Example 3 is detailed in Table 3.

TABLE 3
Metoprolol Tartrate Oral Formulation

| Sr. No. | Excipients | % w/v |
|---|---|---|
| 1 | Metoprolol tartrate* | 1.0 |
| 2 | Sodium benzoate | 0.20 |
| 3 | Xanthan gum | 0.10 |
| 4 | Glycerine | 10.0 |
| 5 | Propylene glycol | 0.5 |
| 6 | Sucralose | 0.10 |
| 7 | Disodium Edetate | 0.05 |
| 8 | Citric acid monohydrate ** | 0.20 |
| 9 | Purified water | q.s. to 100% |

*Calculate the Metoprolol tartrate quantity as per quantity required.
** Citric Acid Monohydrate (20% Solution) additional quantity may be required to get the pH in desired range.

Process of Preparation—Metoprolol Oral Solution was prepared by process as discussed in Example 1.

### Example 4. Metoprolol Tartrate Oral Solution 10 mg/mL (F4)

The formulation of Example 4 is detailed in Table 4.

TABLE 4
Metoprolol Tartrate Oral Formulation

| Sr. No. | Excipients | % w/v |
|---|---|---|
| 1 | Metoprolol tartrate* | 1.0 |
| 2 | Sodium benzoate | 0.14 |
| 3 | Xanthan gum | 0.10 |
| 4 | Glycerine | 10.0 |
| 5 | Sucralose | 0.10 |
| 6 | Disodium Edetate | 0.05 |
| 7 | Citric acid monohydrate ** | 0.20 |
| 8 | Purified water | q.s. to 100% |

*Calculate the Metoprolol tartrate quantity as per quantity required.
** Citric Acid Monohydrate (20% Solution) additional quantity may be required to get the pH in desired range.

Process of Preparation—Metoprolol Oral Solution was prepared by process as discussed in Example 1.

### Example 5. Metoprolol Tartrate Oral Solutions 10 mg/mL (F5-F10)

Various Metoprolol Tartrate Oral Solutions are presented beneath.

TABLE 5
Metoprolol Tartrate Oral Formulation

| | F5 | F6 | F7 | F8 | F9 | F10 |
|---|---|---|---|---|---|---|
| Ingredients | % w/v | % w/v | % w/v | % w/v | % w/v | % w/v |
| Metoprolol tartrate* | 1.0 | 1.0 | 1.0 | 1.0 | 1.0 | 1.0 |
| Sodium benzoate | 0.20 | 0.20 | 0.20 | 0.16 | 0.15 | 0.18 |
| Xanthan gum | 0.10 | 0.10 | 0.10 | 0.10 | 0.10 | 0.10 |
| Glycerine | 10.0 | 10.0 | 10.0 | 10.0 | 10.0 | 10.0 |
| Sucralose | 0.10 | 0.10 | 0.10 | 0.10 | 0.10 | 0.10 |
| Disodium Edetate | 0.04 | 0.07 | 0.08 | 0.05 | 0.05 | 0.05 |
| Citric acid monohydrate ** | 0.20 | 0.20 | 0.20 | 0.20 | 0.20 | 0.20 |
| Purified water | q.s. to | q.s. to | q.s. to | q.s. to | q.s. to | q.s. to |
| | 100% | 100% | 100% | 100% | 100% | 100% |

*Calculate the Metoprolol tartrate quantity as per quantity required.
** Citric Acid Monohydrate (20% Solution) additional quantity may be required to get the pH in desired range.

Process of Preparation—Metoprolol Oral Solutions are prepared by process as discussed in Example 1.

What is claimed is:

1. A liquid pharmaceutical formulation comprising:
(i) metoprolol or a pharmaceutically acceptable salt thereof;
(ii) a preservative;
(iii) a cosolvent; and
(iv) water;
wherein the pH of the formulation is about 2.5 to about 5.5.

2. The formulation of claim 1, wherein the metoprolol is metoprolol tartrate.

3. The formulation of claim 1 or claim 2, wherein the metoprolol is about 0.1% w/v to about 10% w/v of the formulation.

4. The formulation of claim 3, wherein the metoprolol is about 0.5% w/v to about 7% w/v of the formulation.

5. The formulation of claim 4, wherein the metoprolol is about 0.75% w/v to about 5% w/v of the formulation.

6. The formulation of claim 5, wherein the metoprolol is about 0.75% w/v to about 3% w/v of the formulation.

7. The formulation of any one of claims 1 to 6, wherein the preservative comprises sodium benzoate, methyl paraben, propylparaben, ethyl paraben, butyl paraben, sorbic acid, potassium sorbate, sodium sorbate, or a combination thereof.

8. The formulation of any one of claims 1 to 7, wherein the preservative is about 0.01% w/v to about 15% w/v of the formulation.

9. The formulation of claim 8, wherein the preservative is about 0.02% w/v to about 10% w/v of the formulation.

10. The formulation of claim 9, wherein the preservative is about 0.05% w/v to about 5% w/v of the formulation.

11. The formulation of any one of claims 1 to 6, wherein the preservative comprises sodium benzoate.

12. The formulation of claim 11, wherein sodium benzoate is about 0.01% w/v to about 15% w/v of the formulation.

13. The formulation of claim 12, wherein sodium benzoate is about 0.02% w/v to about 10% w/v of the formulation.

14. The formulation of claim 13, wherein sodium benzoate is about 0.05% w/v to about 5% w/v of the formulation.

15. The formulation of any one of claims 1 to 14, wherein the cosolvent comprises propylene glycol, glycerine, or combinations thereof.

16. The formulation of any one of claims 1 to 15, wherein the cosolvent is about 1% w/v to about 50% w/v of the formulation.

17. The formulation of claim 16, wherein the cosolvent is about 2% w/v to about 40% w/v of the formulation.

18. The formulation of claim 17, wherein the cosolvent is about 3% w/v to about 30% w/v of the formulation.

19. The formulation of any one of claims 1 to 14, wherein the cosolvent comprises glycerine.

20. The formulation of claim 19, wherein the glycerine is about 1% w/v to about 50% w/v of the formulation.

21. The formulation of claim 20, wherein the glycerine is about 2% w/v to about 40% w/v of the formulation.

22. The formulation of claim 21, wherein the glycerine is about 3% w/v to about 30% w/v of the formulation.

23. The formulation of any one of claims 1 to 22, further comprising a chelating agent.

24. The formulation of claim 23, wherein the chelating agent comprises disodium edetate, tetrasodium edetate, dipotassium edetate, calcium disodium edetate, L-cysteine, N-acetylL-cysteine, or a combination thereof.

25. The formulation of claim 23 or 24, wherein the chelating agent is about 0.001% w/v to about 10% w/v of the formulation.

26. The formulation of claim 25, wherein the chelating agent is about 0.005% w/v to about 5% w/v of the formulation.

27. The formulation of claim 26, wherein the chelating agent is about 0.01% w/v to about 2% w/v of the formulation.

28. The formulation of claim 23, wherein the chelating agent comprises disodium edetate.

29. The formulation of claim 28, wherein the disodium edetate is about 0.001% w/v to about 10% w/v of the formulation.

30. The formulation of claim 29, wherein the disodium edetate is about 0.005% w/v to about 5% w/v of the formulation.

31. The formulation of claim 30, wherein the disodium edetate is about 0.01% w/v to about 2% w/v of the formulation.

32. The formulation of any one of claims 1 to 31, further comprising a sweetener.

33. The formulation of claim 32, wherein the sweetener comprises sucralose, acesulfame potassium, sodium saccharin, or a combination thereof.

34. The formulation of claim 33, wherein the sweetener is about 0.01% w/v to about 15% w/v of the formulation.

35. The formulation of claim 34, wherein the sweetener is about 0.02% w/v to about 10% w/v of the formulation.

36. The formulation of claim 35, wherein the sweetener is about 0.05% w/v to about 8% w/v of the formulation.

37. The formulation of any one of claims 1 to 36, wherein the formulation is stable for greater than 12 months.

38. The formulation of any one of claims 1 to 36, wherein the formulation is stable for greater than 18 months.

39. The formulation of any one of claims 1 to 38, wherein the formulation does not comprise a buffer.

40. A liquid pharmaceutical formulation comprising:
(i) about 0.1% w/v to about 10% w/v metoprolol tartrate;
(ii) about 0.01% w/v to about 15% w/v preservative;
(iii) about 1% w/v to about 50% w/v cosolvent; and
(iv) water;
wherein the pH of the formulation is about 2.5 to about 5.5.

41. A liquid pharmaceutical formulation comprising:
(i) about 0.1% w/v to about 10% w/v metoprolol tartrate;
(ii) about 0.01% w/v to about 15% w/v sodium benzoate;
(iii) about 1% w/v to about 50% w/v glycerine; and
(iv) water;
wherein the pH of the formulation is about 2.5 to about 5.5.

42. A liquid pharmaceutical formulation comprising:
(i) about 0.1% w/v to about 10% w/v metoprolol tartrate;
(ii) about 0.01% w/v to about 15% w/v preservative;
(iii) about 0.001% w/v to about 10% w/v chelating agent;
(iv) about 0.01% w/v to about 15% w/v sweetener;
(v) about 1% w/v to about 50% w/v cosolvent; and
(v) water;
wherein the pH of the formulation is about 2.5 to about 5.5.

43. A liquid pharmaceutical formulation comprising:
(i) about 0.1% w/v to about 10% w/v metoprolol tartrate;
(iii) about 0.01% w/v to about 15% w/v sodium benzoate;
(ii) about 0.001% w/v to about 10% w/v disodium edetate;
(iv) about 0.01% w/v to about 15% w/v sucralose;
(v) about 1% w/v to about 50% w/v; and
(v) water;
wherein the pH of the formulation is about 2.5 to about 5.5.

44. A liquid pharmaceutical formulation comprising:
(i) about 0.1% w/v to about 10% w/v metoprolol tartrate;
(ii) about 0.01% w/v to about 15% w/v preservative;
(iii) about 0.001% w/v to about 10% w/v chelating agent;
(iv) about 0.01% w/v to about 15% w/v sweetener;
(v) about 0.001% w/v to about 10% w/v thickening agent;
(vi) about 0.001% w/v to about 5% w/v pH adjuster;
(vii) about 1% w/v to about 50% w/v cosolvent; and
(viii) water
wherein the formulation does not comprise a buffer, and wherein the pH of the formulation is about 2.5 to about 5.5.

45. A liquid pharmaceutical formulation comprising:
(i) about 0.1% w/v to about 10% w/v metoprolol tartrate;
(ii) about 0.01% w/v to about 15% w/v sodium benzoate;
(iii) about 0.001% w/v to about 10% w/v disodium edetate;
(iv) about 0.01% w/v to about 15% w/v sucralose;
(v) about 0.001% w/v to about 10% w/v xanthan gum;
(vi) about 0.001% w/v to about 5% w/v citric acid monohydrate;
(vii) about 1% w/v to about 50% w/v glycerine; and
(viii) water
wherein the formulation does not comprise a buffer, and wherein the pH of the formulation is about 2.5 to about 5.5.

46. The formulation of any one of claims 40 to 45, wherein the formulation is stable for at least 12 months.

47. The formulation of any one of claims 40 to 45, wherein the formulation is stable for at least 18 months.

48. The formulation of any one of claims 40 to 45, wherein the formulation is stable for at least 24 months.

49. The formulation of any one of claims 40 to 45, wherein the formulation is stable for at least 30 months.

50. The formulation of any one of claims 40 to 45, wherein the formulation is stable for at least 36 months.

51. The formulation of any one of claims 1 to 50, wherein the formulation does not have more than about 5% w/w of total degradation products at the end of shelf life.

52. The formulation of any one of claims 1 to 51, wherein the formulation does not have more than about 0.5% w/w of specified impurity Metoprolol Related Compound C at the end of shelf life.

53. The formulation of any one of claims 1 to 45, wherein the shelf life of the formulation is 12 months, 18 months, 24 months, 30 months or 36 months.

54. A method of preparing stable liquid oral formulations of the present disclosure comprising
(i) heating purified water in a first vessel to 65-85° C. and adding chelating agent while stirring until a clear solution is formed, and allowing the solution to cool to 25-35° C.;
(ii) heating purified water in a second vessel to 40 C° to 45° C. and adding thickening agent while stirring until a clear solution is formed, and allowing the solution to cool to 25-35° C.;
(iii) adding a cosolvent to the solution of (i) under stirring and mixing for 10 to 15 minutes or until clear solution is formed;
(iv) adding a preservative to purified water in a third vessel under stirring, mixing for 10 to 15 minutes or until clear solution is formed and adding to the solution of (i) under stirring and mixing for 10 to 15 minutes or until clear solution is formed;
(v) adding metoprolol tartrate to purified water in a fourth vessel under stirring and mixing for 10 to 15 minutes or until clear solution is formed;
(vi) adding the solution of (v) to the solution of (i) to which the solution of (ii) and (iv) has already been added under stirring and mixing for 10 to 15 minutes or until clear solution is formed;
(vii) adding a sweetener to water in a fifth vessel under stirring and mixing for 10 to 15 minutes or until clear solution is formed;
(viii) adding the solution of (vii) to the solution of (i) to which the solutions of (ii), (iv) and (v) have been added, under stirring and mixing for 10 to 15 minutes or until clear solution is formed;
(ix) adding a pH adjuster to purified water in a sixth vessel under stirring and mixing for 5-10 minutes or until clear solution is formed;
(x) adding the solution of (ix) to the solution of (i) to which the solutions of (ii), (iv), (v), and (vii) have been added and adjusting the pH of the solution to be between 3.8 to 4.2 with the solution of (ix) as needed; and
(xi) adjusting the volume of the solution of (x) using purified water in quantity sufficient to make up the volume to 100%.

55. A method of treating a disease or condition responsive to beta-blockers in a subject in need thereof, the method comprising administering a liquid pharmaceutical formulation comprising:
(i) metoprolol or a pharmaceutically acceptable salt thereof;
(ii) a preservative;
(iii) a cosolvent; and
(iv) water;
wherein the formulation does not comprise a buffer, and wherein the pH of the formulation is about 2.5 to about 5.5.

56. The method of claim 55, wherein the disease or condition responsive to beta-blockers comprises angina, hypertension, or myocardial infarction.
